SEQUENCE LISTING (54 sequences)

SEQ ID NO: 1 - DNA, 238 bp, Procambarus virginalis
atacagcgagcaatgcgagtacttgaccttatgaggcatcaagaaactcacaaaataggggtattgtatgtggctcaaaatcaaacttcagaacaagaaattttaaggaattcatgtggttcactgcgttacatgcatttccttcagggtttaggtacagtccttgagctgaactctgtatcacaagatgaagtattcctcggtggtcttgacaccaagggtaacgatggcaagctag

SEQ ID NO: 2 - DNA, 248 bp, Procambarus virginalis
aaacgatcggacttggattttatctttggcaaacttataggagaaggaagtttctcaagcgtttaccttgcaaaggacatacacacaaatcaggaatatgcagttaaggtttgtgaaaagcagttaattatacgggagaagaaagtgcagcaaataaccagggaaagggatgtaatgaacctactcaacagcaaccagaaccctacggctccgtttttcgttaaactttcttacgccttccaaggaga

SEQ ID NO: 3 - DNA, 282 bp, Procambarus virginalis
ggtggtccaggtgatgtaagagaggttcagagtcatcccttctatgtaacaatcaactggaaacttcttgaagaaaaaaagttaactccaccattcaagccacaagtaaccagcgagactgacacccggtacttcgatcgagaattcactggagagtctgtgcagcttactccacctgatcaaggggagcaccttaatgttattgatgaagaatcagaatacttgactttcaaccacttctcttatcaggacattttatcaactcttggcagctcactagca

SEQ ID NO: 4 - DNA, 200 bp, Procambarus virginalis
cccatgtcccctggtataggtgggtggggtggcgagtactggcctcaccatgctcaccaacatccacacccgcacgaccgctatgccgaccaactggtagactccatgggggagggactcaagctaggaccggactcttggggtggccctgctcgtccgcccaaccatcaggactgtatgaaactatcccagctctcccc

SEQ ID NO: 5 - DNA, 292 bp, Procambarus virginalis
caatggcatgtttggatgatgaatactcgtggagcccgccttcagaagcggaactcaaagttattgaggccagacgggaacgtaacaacaagatatcatccataatgggacaatatcttctaaagggatacaaaatgttggctataacatgcccagtttgcgagtgcattttgttagaggatcgcatacaaaataaatattgcatcggatgcagtgaagttgatgctgacacatggaaggacaatccagcggttagtgaagaagcagccagaagagcagtggaagaaattca

SEQ ID NO: 6 - DNA, 220 bp, Artificial Sequence
  pAcGFP-N1
cacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatg

SEQ ID NO: 7 - DNA, 267 bp, Procambarus virginalis
  misc_feature (49)..(49): n is a, c, g, or t
  misc_feature (171)..(171): n is a, c, g, or t
ccctgaatcgacaccctttactcaccaataaagatcggaagccagtaangttggcagtcgccgaactgttgctgcattgtgtagccttaaaactcaacacaaatgtaggtaaggacaacagagccatcgtctgcagagtttagaggccagttctaggtcctttgtcacctntaaaaaggaacaaaccccattcagttttcctgatcagtgccaagacttgtttaatagagtggaagcgatctaccctcctccaaagtaagttgcagc

SEQ ID NO: 8 - DNA, 229 bp, Procambarus virginalis
tcaagattctcaaccgcaaaactatcaagaatttggatatggtcagcaagataaaacgagaaataacaaatcttaaattgtttcgtcatccacatatcattaaactgtaccaggtgatcagcacacctacagatatctttatggtgatggaatatgcttcaggaggagagctttttgactatattaagaaaaaaggaaagctgaaggaatctgaagctcgcaggttctt

SEQ ID NO: 9 - DNA, 319 bp, Procambarus virginalis
tggctacgacctggatctcagctatatcacagatcgtcttatcgccatgggcttccctgctcagaagttggagggtgtctacagaaaccatattgatgacgtatgccgcttcctagaagacagacacaaggaccattatagaatatataatttgtgttctgagagaaatcgatcgtacgacgtagcaagattccataaccgcgttagaacgttcccatttgctgaccacaatccacctcctctgattgatatcgagccactatgcaaagatatggcagattggctcaatgaagatcagaaaaatgtaggctgttgtgca

SEQ ID NO: 10 - DNA, 433 bp, Procambarus virginalis
ccagacctggagaggtttcataccttaagcttggtgctgagttaccagtaagagaagaaggttctacgagttgcttgtggaagagcaccagagcgggtgtcagtagtgcttcaagacatggttaaccaagctgctcaatgcttcattgtacggagagtgtggctggtggtggtggttgggctgctggtacaccagacagcggcaaggtatgtcttcgaagaatgtccaggagtgatgggcaaccgagccgtccacggcaaggtgacccgggtttgtgaggattgctacaacgtcttcagggacactgaagtcttggctggatgcaggaaaggctgcttttctagtgagatgttcaagctttgcctcttggctatggagcgcgtcgaggagtttccagacttcaagagatggattggtattcttaacgccggtc

SEQ ID NO: 11 - DNA, 25 bp, Artificial Sequence
  Forward primer for Akt
ccgtgacctgcttaaaggtttgtta

SEQ ID NO: 12 - DNA, 25 bp, Artificial Sequence
  Reverse primer for Akt
tctcttacatcacctggaccacctc

SEQ ID NO: 13 - DNA, 25 bp, Artificial Sequence
  Forward primer for FOXO
tgagtgagagtctggacctgtaccc

SEQ ID NO: 14 - DNA, 20 bp, Artificial Sequence
  Reverse primer for FOXO
gctgaaggcagggtggactg

SEQ ID NO: 15 - DNA, 25 bp, Artificial Sequence
  Forward primer for PDK
gcaaataaccagggaaagggatgta

SEQ ID NO: 16 - DNA, 25 bp, Artificial Sequence
  Reverse primer for PDK
agtttaacgaaaaacggagccgtag

SEQ ID NO: 17 - DNA, 25 bp, Artificial Sequence
  Forward primer for PI3K
attgctacaaacaccgatctgccta

SEQ ID NO: 18 - DNA, 25 bp, Artificial Sequence
  Reverse primer for PI3K
ctcatcgtgtaatctgtccgtgatg

SEQ ID NO: 19 - DNA, 25 bp, Artificial Sequence
  Forward primer for PTEN
agcaagattccataaccgcgttaga

SEQ ID NO: 20 - DNA, 23 bp, Artificial Sequence
  Reverse primer for PTEN
gattgtggtcagcaaatgggaac

SEQ ID NO: 21 - DNA, 25 bp, Artificial Sequence
  Forward primer for AMPK
attaccatcaacgagagcgactttg

SEQ ID NO: 22 - DNA, 25 bp, Artificial Sequence
  Reverse primer for AMPK
gagactgactgctcaacggcttatc

SEQ ID NO: 23 - DNA, 25 bp, Artificial Sequence
  Forward primer for mTOR
acattcacgcttcaagagaccaaag

SEQ ID NO: 24 - DNA, 25 bp, Artificial Sequence
  Reverse primer for mTOR
gtcaccttcttgattgtggctgagt

SEQ ID NO: 25 - DNA, 25 bp, Artificial Sequence
  Forward primer for PRAS40
ggcatagcagagggacagaagtttt

SEQ ID NO: 26 - DNA, 25 bp, Artificial Sequence
  Reverse primer for PRAS40
atttggtggcagttcagacactctc

SEQ ID NO: 27 - DNA, 25 bp, Artificial Sequence
  Forward primer for Raptor
aagaatggaggcagaaaggcagtag

SEQ ID NO: 28 - DNA, 25 bp, Artificial Sequence
  Reverse primer for Raptor
tccacaggctccaagttgaatacat

SEQ ID NO: 29 - DNA, 25 bp, Artificial Sequence
  Forward primer for Rheb
agaagctgaaggttagagggcaaga

SEQ ID NO: 30 - DNA, 25 bp, Artificial Sequence
  Reverse primer for Rheb
tccagccgtgtctactaactccaag

SEQ ID NO: 31 - DNA, 25 bp, Artificial Sequence
  Forward primer for S6K1
cgataacggtgatccaatcaagaaa

SEQ ID NO: 32 - DNA, 25 bp, Artificial Sequence
  Reverse primer for S6K1
ggtttaaatggcggttctaacttgc

SEQ ID NO: 33 - DNA, 25 bp, Artificial Sequence
  Forward primer for TSC1
tgtgaggaggtatgtggctcatttt

SEQ ID NO: 34 - DNA, 25 bp, Artificial Sequence
  Reverse primer for TSC1
attggtggacaaattcaaaggttcc

SEQ ID NO: 35 - DNA, 25 bp, Artificial Sequence
  Forward primer for TSC2
gaagtattcctcggtggtcttgaca

SEQ ID NO: 36 - DNA, 25 bp, Artificial Sequence
  Reverse primer for TSC2
tacgtggaaaacaacctgcataacc

SEQ ID NO: 37 - DNA, 25 bp, Artificial Sequence
  Forward primer for 4EBP
agaacctgccagtgataccaggagt

SEQ ID NO: 38 - DNA, 25 bp, Artificial Sequence
  Reverse primer for 4EBP
ctcctctgaaatcgttccattttcc

SEQ ID NO: 39 - DNA, 25 bp, Artificial Sequence
  Forward primer for FGF1
acatctacgccatcctggaggttcg

SEQ ID NO: 40 - DNA, 25 bp, Artificial Sequence
  Reverse primer for FGF1
aagagcccagcatcaacacctctga

SEQ ID NO: 41 - DNA, 25 bp, Artificial Sequence
  Forward primer for ILP
caccttggctgatattcaaagatgg

SEQ ID NO: 42 - DNA, 25 bp, Artificial Sequence
  Reverse primer for ILP
ttgccagagattgtttgagttctcc

SEQ ID NO: 43 - DNA, 25 bp, Artificial Sequence
  Forward primer for E75
cgggaggcacttcatacatctgtta

SEQ ID NO: 44 - DNA, 25 bp, Artificial Sequence
  Reverse primer for E75
agtcaaacatcgagtccatcaggaa

SEQ ID NO: 45 - DNA, 25 bp, Artificial Sequence
  Forward primer for EcR
tcaagaatgtcgcctgaagaaatgt

SEQ ID NO: 46 - DNA, 25 bp, Artificial Sequence
  Reverse primer for EcR
ttctgttcacgttttacctggcatt

SEQ ID NO: 47 - DNA, 25 bp, Artificial Sequence
  Forward primer for Kr-h1
tcgcctctttcaagtaattcagacg

SEQ ID NO: 48 - DNA, 25 bp, Artificial Sequence
  Reverse primer for Kr-h1
atattgaggctggtgaggagttggt

SEQ ID NO: 49 - DNA, 25 bp, Artificial Sequence
  Forward primer for Met
ttggccacagtcaggttgacttatt

SEQ ID NO: 50 - DNA, 25 bp, Artificial Sequence
  Reverse primer for Met
atcgggatgaatgacgttgtagatg

SEQ ID NO: 51 - DNA, 23 bp, Artificial Sequence
  Forward primer for MIH
ttgctacaacgtcttcagggaca

SEQ ID NO: 52 - DNA, 23 bp, Artificial Sequence
  Reverse primer for MIH
catagccaagaggcaaagcttga

SEQ ID NO: 53 - DNA, 20 bp, Artificial Sequence
  Forward primer for EF-1alpha
gcagattgtgccgtgctgat

SEQ ID NO: 54 - DNA, 19 bp, Artificial Sequence
  Reverse primer for EF-1alpha
ctcgggtctgaccgtgctt

(19) United States
(12) Patent Application Publication
(10) Pub. No.: US 2023/0397582 A1
(43) Pub. Date: Dec. 14, 2023

(54) METHOD FOR REGULATING GROWTH OF DECAPODA CRUSTACEANS

(71) Applicants: The University of Tokyo, Tokyo (JP); NH Foods Ltd., Osaka (JP)

(72) Inventors: Junpei SHINJI, Tokyo (JP); Taro YONEKITA, Ibaraki (JP)

(73) Assignees: The University of Tokyo, Tokyo (JP); NH Foods Ltd., Osaka (JP)

(21) Appl. No.: 18/033,987

(22) PCT Filed: Oct. 27, 2021
(86) PCT No.: PCT/JP2021/039717

§ 371 Date: Apr. 26, 2023

(30) Foreign Application Priority Data
Oct. 27, 2020 (JP) 2020-179947

(51) Int. Cl. A01K 67/033; C12N 15/113

(52) U.S. Cl. CPC A01K 67/0338; C12N 15/113

(57) ABSTRACT

The present invention provides a means for regulating (promoting or suppressing) the growth of Decapoda crustaceans such as shrimps and crabs. Methods of the present invention regulate the growth of animals belonging to the Decapoda, comprising a step of regulating (inhibiting or enhancing) the function of genes comprising: at least one growth regulation-related gene selected from the group consisting of mTOR pathway, Akt pathway, and upstream and downstream factors of the pathways; and further optionally at least one molting-related gene selected from a molting-related factor or the function of a transcription or translation product of the genes.

### TECHNICAL FIELD

The present invention relates to methods for regulating the growth of Decapoda crustaceans (such as shrimps and crabs), particularly methods for promoting the growth of Decapoda crustaceans. More particularly, the present invention relates to methods for regulating the growth of Decapoda crustaceans, particularly methods for promoting the growth of Decapoda crustaceans, comprising inhibiting or enhancing the function of specific genes or transcription or translation products thereof.

### BACKGROUND ART

Japan has long had the highest demand for Decapoda crustaceans such as shrimps and crabs in the world, and the world's first full cycle aquaculture of shrimps has been established in Japan. Recently, the demand for shrimps and other crustaceans has been rapidly increasing on a global scale, and aquaculture of shrimps and other crustaceans has been attracting attention as growth industry worldwide. However, there are no breeds established for shrimp aquaculture in contrast with the livestock industry, which has many breeds specialized for meat production. To meet the ever-increasing production demands, the aquaculture is forced to perform under high-density conditions that do not naturally occur, leading to problems continuing from the dawn of shrimp aquaculture including reduced growth rates and high mortality. Aquaculture of shrimps and other crustaceans has still room for highly improved productivity by generating breeds that grow faster and grow to be stronger and larger or by developing methods for growing shrimps and other crustaceans faster and growing them to be stronger and larger. On the other hand, there is also a need for methods of suppressing the growth, for example, to make detrimental Decapoda crustaceans smaller and more easily eaten by foreign enemies or to reduce energy used for growth to promote sexual maturation.

So far, methods for increasing the growth rate of Decapoda crustaceans such as shrimps and crabs have been proposed, including methods of administering low- and/or high-molecular-weight lignin (Patent Literature 1), methods of administering a peptide (GHRP-6) having a predetermined amino acid sequence (Patent Literature 2), and methods of administering a fatty acid ester of cholesterol represented by a predetermined general formula (Patent Literature 3). On the other hand, although it has been reported that suppressing the expression of Molt-Inhibiting Hormone (MIH) gene of Macrobrachium nipponense by RNAi allows acceleration of molting cycles (Non-Patent Literature 1), methods for increasing the growth rate of Decapoda crustaceans by altering (suppressing or enhancing) gene expression have been little known.

### CITATION LIST

### Patent Literature

Patent Literature 1: WO 2018/079641 (Japanese Patent No. 6344534)

Patent Literature 2: WO 2003/080102 (Japanese Patent No. 4195865)

Patent Literature 3: JP 1997(H09)-084527

### Non Patent Literature

Non Patent Literature 1: Qial et al. PLoS One. 2018; 13 (6): e0198861.

### SUMMARY OF INVENTION

### Technical Problem

Conventional methods, such as those described in Patent Literatures 1 to 3 and Non-Patent Literature 1, have room for improvement in effects of growth promotion (such as persistence and stability).

The present invention aims to provide means for regulating the growth of animals belonging to the Decapoda (Decapoda crustaceans), such as shrimps and crabs, for example, means for promoting growth, particularly comprising altering gene expression.

### Solution to Problem

The present inventors have focused on genes that mediate the transmission of environmental information, such as Tuberous Sclerosis Complex (TSC) 1/TSC2, among genes in the mTOR signaling pathway (sometimes herein referred to as the “mTOR pathway”) involved in the control of environmental information that affects the growth of organisms. As disclosed in the Examples described below, the present inventors confirmed excellent growth-promoting effects of TSC2 and other genes by inhibiting functions of these genes in marbled crayfish, which is a model organism of Decapoda crustaceans, by siRNA-based RNAi. The present inventors also have focused on AMP-activated protein kinase (AMPK), which is activated in response to less energy available to among genes in the mTOR signaling pathway and confirmed that inhibiting the functions of these genes also produces an excellent growth-promoting effect. The present inventors further analyzed the growth of individuals in Decapoda crustaceans (marbled crayfish) and the expression levels of upstream and downstream genes (factors) of pathways including the mTOR signaling pathway, PDK1-Akt signaling pathway (sometimes herein referred to as the “Akt pathway”), and other growth-related signaling pathways, such as involving molting-related factors, by principal component analysis. As a result, the present inventors found a group of genes that can be interpreted as growth-promoting when their expression is suppressed or conversely as growth-suppressing when their expression is enhanced and another group of genes that can be interpreted as growth-promoting when their expression is enhanced or conversely as growth-suppressing when their expression is suppressed. The present inventors have come up with a comprehensive technical idea based on these findings and finally completed the present invention that relates to the mTOR pathway, Akt pathway, and upstream factors of these pathways, molting-related factors, and regulation of growth in Decapoda crustaceans in the direction of both promotion and inhibition.

Thus, in one aspect, the present invention provides the following clauses:

### [Clause 1]

A method for regulating growth of an animal belonging to the Decapoda, comprising a step of regulating function of genes comprising: at least one growth regulation-related gene selected from the group consisting of mTOR pathway, Akt pathway, and upstream and downstream factors of the pathways; and further optionally at least one molting-related gene selected from a molting-related factor, or function of a transcription or translation product of the genes.

### [Clause 2]

The method according to clause 1, wherein the genes comprise at least one growth regulation-related gene selected from the group consisting of Akt, AMPK, FOXO, p27, PDK, PTEN, TBC1D7, TSC1, and TSC2.

### [Clause 3]

The method according to clause 1, wherein the genes comprise at least one molting-related gene selected from the group consisting of EcR, Kr-hl, Met, and MIH.

### [Clause 4]

The method according to any one of clauses 1 to 3, wherein the genes comprise, as the growth regulation-related gene, at least one growth regulation-related gene selected from the group consisting of AMPK, TSC1, TSC2, and PDK, and wherein the step comprises inhibiting the function of the growth regulation-related gene or a transcription or translation product thereof for regulation of the function, to promote the growth of the animal belonging to the Decapoda.

### [Clause 5]

The method according to clause 4, wherein the growth regulation-related gene comprises at least AMPK and TSC1 and/or TSC2.

### [Clause 6]

The method according to any one of clauses 1 to 3, wherein the genes comprise at least Akt as the growth regulation-related gene, and wherein the step comprises enhancing the function of the growth regulation-related gene or a transcription or translation product thereof for regulation of the function, to promote the growth of the animal belonging to the Decapoda.

### [Clause 7]

The method according to any one of clauses 1 to 3, wherein the genes comprise at least Akt as the growth regulation-related gene, and wherein the step comprises inhibiting the function of the growth regulation-related gene or a transcription or translation product thereof for regulation of the function, to suppress the growth of the animal belonging to the Decapoda.

### [Clause 8]

The method according to any one of clauses 1 to 3, wherein the genes comprise: at least PTEN as the growth regulation-related gene; and at least MIH as the molting-related gene, and wherein the step comprises inhibiting the function of the growth regulation-related gene and molting-related gene or a transcription or translation product thereof for regulation of the function, to promote the growth of the animals belonging to the Decapoda.

### [Clause 9]

The method according to clause 1, wherein the genes comprise: at least one growth regulation-related gene selected from the group consisting of 4EBP, Akt, FGF1, FOXO, ILP, PTEN, Rheb, S6K1, TSC1, and TSC2; and further optionally at least one molting-related gene selected from the group consisting of EcR, Kr-hl, Met, and MIH.

### [Clause 10]

The method according to any one of clauses 1 to 5 and 7 to 9, wherein the regulating the function of the genes or the transcription or translation product thereof is to inhibit the function by suppressing the expression of the genes by RNA interference (RNAi), an antisense method, or genome editing.

### [Clause 11]

An animal belonging to the Decapoda, having regulated function of genes comprising: at least one growth regulation-related gene selected from the group consisting of mTOR pathway, Akt pathway, and upstream and downstream factors of the pathways; and further optionally at least one molting-related gene selected from a molting-related factor, or function of a transcription or translation product thereof.

### [Clause 12]

The animal according to clause 11, wherein the genes comprise at least one growth regulation-related gene selected from the group consisting of Akt, AMPK, FOXO, p27, PDK, PTEN, TBC1D7, TSC1, and TSC2.

### [Clause 13]

The animal according to clause 11, wherein the genes comprise at least one molting-related gene selected from the group consisting of EcR, Kr-hl, Met, and MIH.

### [Clause 14]

The animal according to clause 11, wherein the genes comprise: at least one growth regulation-related gene selected from the group consisting of 4EBP, Akt, FGF1, FOXO, ILP, PTEN, Rheb, S6K1, TSC1, and TSC2; and further optionally at least one molting-related gene selected from the group consisting of EcR, Kr-hl, Met, and MIH.

### [Clause 15]

The animal according to any one of clauses 11 to 14, wherein the regulating the function of the genes or the transcription or translation product thereof is inhibition, and the animal harbors a double-stranded RNA or vector for suppressing the expression of the genes by RNA interference (RNAi), a chromosome having a loss of function of the genes, or an inhibitor for the translation product of the gene or a receptor thereof, in the body.

### Advantageous Effects of Invention

The methods for regulating the growth of Decapoda crustaceans according to the present invention can be utilized to promote the growth even in large-scale rearing environments such as aquaculture or conversely to suppress the growth depending on the application purposes. For example, knockdown of a specific gene without genetic modification, which is preferable in view of environmental conservation, allows generation of high-growth Decapoda crustacean individuals, and genetic modification of a specific gene can generate high-growth Decapoda crustacean strains having effects of the gene that will be passed on to the next generation.

### BRIEF DESCRIPTION OF DRAWINGS

FIG. 1 shows the results for the difference in growth between the TSC2 function-inhibiting group (TSC2^{RNAi}) and the control group of inhibiting GFP function (GFP^{RNAi}) in Example 1. [A] Body size of individuals in the TSC2 function-inhibiting group (25 days after the start) was compared with that of individuals in the control group (31 days after the start) (the individual body weight at the start was 28 mg in both groups). [B] The progression of individual body weights versus the number of days after the start of the experiment is shown. On the basis of the results of individual body weights measured one day after molting, the relationship between the individual body weights and the elapsed number of days was analyzed by linear regression analysis. As a result, the regression coefficient, which indicates the growth rate per day, was approximately twice as high in the TSC2 function-inhibiting group as in the control group. [C] The progression of increasing rate of individual body weight per molting is shown. [D] The progression of intervals between moltings per molting is shown. [E] The progression of individual body weights per molting is shown. In the TSC2 function-inhibiting group, the amounts of growth through molting were increased, and molting was accelerated by one molting within the experimental period. *:n=7 to 9; Students' t-test or Welch's t-test; P<0.05.

FIG. 2 shows photographs comparing the body size of an individual of the TSC1 and AMPK function-inhibiting group (TSC2^{RNAi} & AMPK^{RNAi}) (19 days after the start) and of the control group (GFP^{RNAi}) (16 days after the start) in Example 2 (the body weights of both individuals at the start were 20 mg).

FIG. 3 shows a schema of mTOR signaling pathway (cited from Journal of Cell Science 122, 3589-3594. doi:10.1242/jcs.051011).

FIG. 4 shows the relationship between the principal component scores of post-molting growth PC1 predicted from the pre-molting state of the individuals using the mathematical model established in Example 4 and the principal component scores of actual post-molting growth PC1.

FIG. 5 shows the correlation between gene expression levels and growth PC1 obtained in Example 4. [A] Correlation with candidate upstream ligand factors of Akt pathway and mTOR pathway is shown. Significant correlations were observed for only two factors, FGF1 and ILP, surrounded by the dashed line. [B] Among factors in the mTOR pathway and Akt pathway, the gene expression kinetics of Rheb, which has correlated particularly highly with growth PC1, is shown as an example.

FIG. 6 shows a path diagram model showing the relationship between the expression kinetics of each gene and growth, created on the basis of the results listed in Tables 6 and 7 in Example 4.

### DESCRIPTION OF EMBODIMENTS

Methods for Regulating (Promoting or Suppressing) Growth

Methods for regulating the growth of animals belonging to the Decapoda (sometimes herein referred to as “subject animals”) (sometimes herein referred to as “growth regulating methods of the present invention”) according to the present invention comprise a step of regulating the function of a specific gene or a transcription or translation product thereof (as used herein, the gene and transcription and translation product thereof are sometimes collectively herein referred to as “specific gene, etc.”) (sometimes herein referred to as “regulating step”).

The growth regulating methods of the present invention include (I) a method for promoting the growth of a subject animal (sometimes herein referred to as “growth promoting method of the present invention”) and (II) a method for suppressing the growth of a subject animal (sometimes herein referred to as “growth suppressing method of the present invention”) and may vary depending on embodiments of the present invention or application purposes. The regulating step includes (i) a step of inhibiting the function of a specific gene, etc. (sometimes herein referred to as “inhibiting step”), (ii) a step of enhancing the function of a specific gene, etc. (sometimes herein referred to as “enhancing step”), and (iii) a step of inhibiting the function of a specific gene, etc. and enhancing the function of another specific gene, etc. (sometimes herein referred to as “suppressing/enhancing step”) and may vary depending on embodiments of the present invention. As described in detail below, the specific gene, etc. includes (A) one that promotes the growth of subject animals by inhibiting the function of the genes or conversely that suppresses the growth of subject animals by enhancing the function of the genes and (B) one that suppresses the growth of subject animals by inhibiting the function of the genes or conversely that promotes the growth of subject animals by enhancing the function of the genes. Thus, the growth regulating methods of the present invention include, for example, (I-i) a growth promoting method comprising a step of inhibiting a specific gene, etc., (I-ii) a growth promoting method comprising a step of enhancing a specific gene, etc., (I-iii) a growth promoting method comprising a step of suppressing/enhancing a specific gene, in other words, a step of inhibiting a specific gene, etc. and a step of enhancing another specific gene, etc., (II-i) a growth suppressing method comprising a step of inhibiting a specific gene, etc., (II-ii) a growth suppressing method comprising a step of enhancing a specific gene, etc., and (II-iii) a growth suppressing method comprising a step of suppressing/enhancing a specific gene, etc., in other words, a step of inhibiting a specific gene, etc. and a step of enhancing another specific gene, etc.

The “subject animals” (animals belonging to the Decapoda, Decapoda crustaceans) as used herein is not particularly limited and includes various Decapoda crustaceans that are referred to as shrimps, crabs, hermit crabs, crayfish, and the like. Representative examples of the subject animals in the present invention include Marbled crayfish (Procambarus virginalis), a freshwater decapod that is exceptionally easy to rear and breed among decapod crustaceans, has recently attracted attention as a model organism for Decapoda crustaceans, and was used in the Examples described below. The subject animals are preferably those of high importance in the aquaculture business such as penaeid shrimps (Marsupenaeus japonicus), tiger shrimps (cattle shrimp, Penaeus monodon), and whiteleg shrimps (Penaeus vannamei). Shrimps belonging to the Pandalidae and Nephropidae, swimming crabs, and king crabs, which are the subject animals of fish farming, as well as spiny lobsters are also relatively important subject animals.

The “specific gene” as used herein can include a “growth regulation-related gene”. The “growth regulation-related gene” as used herein refers to a gene that represents “mTOR pathway, Akt pathway, and upstream and downstream factors of these pathways” relating to a regulatory mechanism of cell division or a mechanism directing growth. Examples of the factors of “mTOR pathway” include genes such as 4EBP, AMPK, mTOR, PRAS40, Raptor, Rheb, S6K1, TBC1D7, TSC1, and TSC2. Examples of the factors of “Akt pathway” include genes such as Akt, FOXO, PDK, and PTEN. Examples of the “downstream factors of mTOR pathway and Akt pathway” include a gene that controls cell cycle, such as p27. Examples of the “upstream factors of mTOR pathway and Akt pathway” include genes such as FGF1 and Insulin-like peptide (ILP). The genes of mTOR pathway and upstream factors of mTOR pathway can be found in FIG. 3. The growth regulation-related gene may be any one or a combination of two or more of the genes. The growth regulation-related gene may be at least one gene selected from any one of a “factor of mTOR pathway”, a “factor of Akt pathway”, an “upstream factor of mTOR pathway and/or Akt pathway”, and a “downstream factor of mTOR pathway and/or Akt pathway” or at least two genes (at least one gene for each factor) selected from these factors.

The “specific gene” as used herein can include a “molting-related gene”. In this embodiment, the specific gene may not include a growth regulation-related gene. The “molting-related gene” as used herein refers to a gene that is a “molting-related factor” and relates to a regulatory mechanism of molting. Examples of the molting-related gene include E75, EcR, Kr-hl, Met, and MIH. The molting-related gene may be any one or a combination of two or more. The molting-related gene may be at least one selected from the group consisting of, for example, E75, EcR, Kr-hl, and Met and may be preferably Kr-hl.

In one embodiment of the present invention, the “specific gene” can include a “growth regulation-related gene” and further optionally a “molting-related gene”. In other words, the specific gene may include only at least one growth regulation-related gene or both at least one growth regulation-related gene and at least one molting-related gene.

In one embodiment of the present invention, the “specific gene” can include a “molting-related gene” and further optionally a “growth regulation-related gene”. In other words, the specific gene may include only at least one molting-related gene or both at least one molting-related gene and at least one growth regulation-related gene.

The “specific gene” (sometimes herein referred to as “Specific Gene 0”) in one embodiment of the present invention can include: “at least one gene selected from the group consisting of 4EBP, Akt, AMPK, FGF1, FOXO, ILP, p27, PDK, PTEN, Rheb, S6K1, TBC1D7, TSC1, and TSC2” as a growth regulation-related gene; and further optionally “at least one gene selected from the group consisting of EcR, Kr-hl, Met, and MIH” as a molting-related gene. The Specific Gene 0 comprises “Specific Gene 1” added to “Specific Gene 2”, in which they are described next.

The “specific gene” (sometimes herein referred to as “Specific Gene 1”) in one embodiment of the present invention can include: “at least one gene selected from the group consisting of Akt, AMPK, FOXO, p27, PDK, PTEN, TBCID7, TSC1, and TSC2” as a growth regulation-related gene; and further optionally “at least one gene selected from the group consisting of EcR, Kr-hl, Met, and MIH” as a molting-related gene. Among the growth regulation-related genes included in Specific Gene 1, Akt, FOXO, PDK, and PTEN are factors of the Akt pathway, and p27 is a downstream factor of the mTOR pathway and Akt pathway. AMPK, TBC1D7, TSC1, and TSC2 are factors of the mTOR pathway. Specific Gene 1 can be found in Example 1 (Table 1), Example 2 (Table 2), Example 3 (Table 3), and the like described herein below.

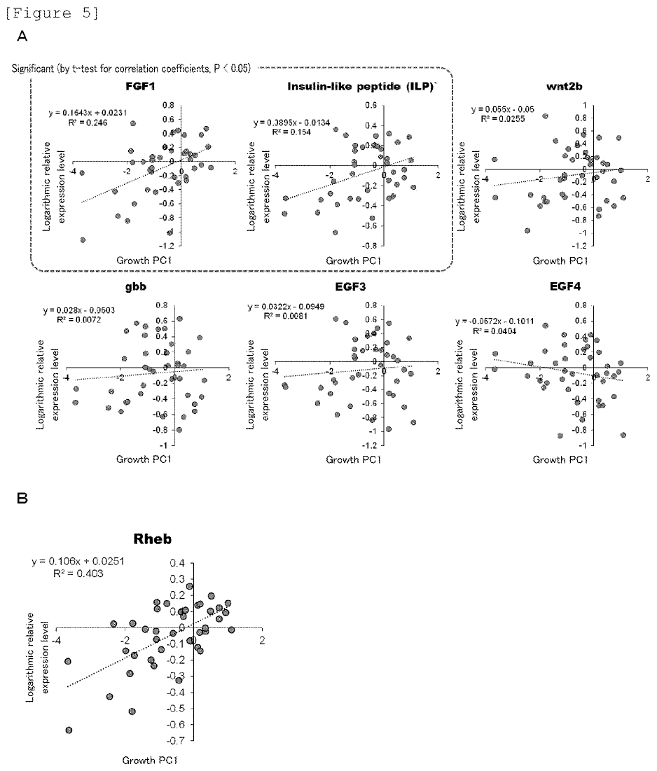

The “specific gene” (sometimes herein referred to as “Specific Gene 2”) in one embodiment of the present invention can include: “at least one gene selected from the group consisting of 4EBP, Akt, FGF1, FOXO, ILP, PTEN, Rheb, S6K1, TSC1, and TSC2” as a growth regulation-related gene; and further optionally “at least one gene selected from the group consisting of EcR, Kr-hl, Met, and MIH” as a molting-related gene. Among the growth regulation-related genes included in Specific Gene 2, Akt, FOXO, and PTEN are factors of the Akt pathway, 4EBP, Rheb, S6Kl, TSC1, and TSC2 are factors of the mTOR pathway, and FGF1 and ILP are upstream factors of the Akt pathway and mTOR pathway. Specific Gene 2 can be found in Example 4 (Table 4 and FIG. 5) and the like described herein below. It should be noted that Akt, FOXO, PTEN, and TSC2 correspond to both Specific Gene 1 and Specific Gene 2 and thus may be discussed as Specific Gene 1 or Specific Gene 2 depending on a technical idea of the present invention.

The specific genes (growth regulation-related gene and molting-related gene) include (A) a gene that promotes the growth of subject animals by inhibiting the function of the gene or conversely that suppresses the growth of subject animals by enhancing the function of the gene (sometimes collectively herein referred to as “Specific Gene A”) and (B) a gene that suppresses the growth of subject animals by inhibiting the function of the gene or conversely that promotes the growth of subject animals by enhancing the function of the gene (sometimes collectively herein referred to as “Specific Gene B”). Specific Gene A includes (A1) a growth regulation-related gene such as AMPK, FOXO, p27, PDK, PTEN, TBC1D7, TSC1, or TSC2 among Specific Gene 1 and (A2) a growth regulation-related gene such as 4EBP, FOXO, PTEN, S6K1, or TSC2 and a molting-related gene such as Kr-hl or NIH (sometimes collectively herein referred to as “Specific Gene 2A”) among Specific Gene 2. Specific Gene B includes (B1) a growth regulation-related gene such as Akt or PTEN among Specific Gene 1 and (B2) a growth regulation-related gene such as Akt, FGF1, ILP, Rheb, or S6K1 and a molting-related gene such as EcR or Met among Specific Gene 2. It should be noted that PTEN and S6K1 correspond to both Specific Gene A and Specific Gene B and thus may be discussed as Specific Gene A or Specific Gene B depending on embodiments (such as the type of other specific genes to be used in combination and the number of molting).

In one embodiment of the present invention, the specific gene preferably comprises at least one growth regulation-related gene selected from the group consisting of AMPK, TSC1, TSC2, and PDK (in other words, the function of at Least AMPK, TSC1, TSC2, or PDK gene or a transcription or translation product thereof, as a specific gene, etc., is preferably regulated) because the effect on growth regulation of subject animals (e.g., the effect of promoting the growth of subject animals upon inhibition of the function) is enhanced. In this embodiment, the specific gene may be only at least one selected from the group consisting of AMPK, TSC1, TSC2, and PDK (i.e., AMPK alone, TSC1 alone, TSC2 alone, PDK alone, or any combination thereof) or a combination of AMPK, TSC1, TSC2, or PDK and any gene except these (e.g., at least one gene selected from the group consisting of Akt, FOXO, p27, PTEN, and TBC1D7 (Specific Gene 1 except AMPK, TSCI, TSC2, and PDK), or Specific Gene 2). This embodiment will be described in Example 1 (Table 1) and Example 2 (Table 2) described herein below.

In one embodiment of the present invention, the specific gene preferably comprises at least growth regulation-related genes AMPK and TSC1 and/or TSC2 (i.e., the function of at least AMPK gene and TSC1 gene and/or TSC2 gene, or a transcription or translation product thereof, as a specific gene, etc., is preferably regulated) because the effect on growth regulation of subject animals (e.g., the effect of promoting the growth of subject animals upon inhibition of the function) is enhanced. In an embodiment in which AMPK, a factor (gene) that mediates reduction of energy available to cells is used in combination with TSC1/TSC2 mediating the transmission of environmental information, the function that regulates (e.g., promotes) the growth rate of subject animals will be more advantageous and thus more preferable. This embodiment will be described in Example 2 (Table 2) described herein below. In this embodiment, the specific gene may be AMPK and TSC1 and/or TSC2 alone or may be a combination of AMPK and TSC1 and/or TSC2 with any gene except these (e.g., at least one gene selected from the group consisting of Akt, FOXO, p27, PDK, PTEN, and TBC1D7 (Specific Gene 1 except AMPK, TSC1, and TSC2) or Specific Gene 2).

In one embodiment of the present invention, the specific gene preferably comprises at least Akt as a growth regulation-related gene (in other words, the function of at least Akt gene or a transcription or translation product thereof as a specific gene, etc. is preferably regulated) in order to regulate the growth of subject animals (e.g., to promote the growth of subject animals upon enhancement of the function or to suppress the growth of subject animals upon inhibition of the function). This embodiment will be described in Example 1 (Table 1) described herein below.

In one embodiment of the present invention, the specific gene preferably comprises at least PTEN or other growth regulation-related genes (preferably included in Specific Gene 2) and MIH or comprises PTEN and MIH or other molting-related genes, in order to regulate the growth of subject animals (e.g., to promote the growth of subject animals upon inhibition of the function). This embodiment will be described in Example 3 described herein below.

Means for regulating (inhibiting or enhancing) the function of a specific gene, etc. are not particularly limited and may be various means commonly used and well known or known. Suitable conditions for any selected means may be determined.

Means for inhibiting the function of a specific gene itself include, for example, genome editing techniques (a CRISPR-Cas system, TALEN, and ZFN) or other gene recombination techniques (such as classical homologous recombination). For example, a CRISPR-Cas system can be used to introduce mutation (insertion, deletion, substitution, and/or addition of at least one nucleotide) into a specific gene on a chromosome resulting in a loss of function of the specific gene by administering appropriate elements for the system employed (such as RNA including crRNA corresponding to a nucleotide sequence of a specific gene, tracrRNA, and sgRNA and a Cas protein (such as Cas9 or Cas3) or mRNA or a vector to express the Cas protein) to individuals, embryos, eggs, and other cells of subject animals by using any suitable techniques (such as injection, electroporation, or addition into culture medium), in combination with an appropriate means for delivering the elements into cells (such as liposomes) if necessary. Such RNAs, vectors, and other necessary elements may be designed and produced according to any conventional method. Some specific genes may have multiple copies of the same gene in the genome (located in a plurality of positions). In such cases, the functions of all of the multiple copies may be lost, or some (at least one) of the multiple copies may be lost.

Means for inhibiting the function of a transcription product (mRNA) of a specific gene include, for example, RNA interference (RNAi) and conventional antisense methods, in which a transcription product of a specific gene is degraded, or the translation of the transcription product into a protein is inhibited. To suppress the expression of a specific gene, RNAi uses (a) an siRNA (synthetic double-stranded RNA) comprising a nucleotide sequence of a part of mRNA of the specific gene and a nucleotide sequence complementary thereto, which can be incorporated into an RNA-induced silencing complex (RISC) to direct degradation of the mRNA of the specific gene, (b) a vector to generate an shRNA, which is a hairpin RNA to provide an siRNA, or (c) a vector to generate an miRNA, which binds to the 3-terminal region of RISC to inhibit the translation of the mRNA of the specific gene into a protein. Conventional antisense methods, on the other hand, use a nucleic acid that comprises a nucleotide sequence complementary to a mRNA of a specific gene. RNAi, antisense methods, or other methods can be used to administer appropriate elements as described above for the method employed to subject animal individuals by appropriate means (such as injection), in combination with any appropriate means for delivering the elements into cells (such as liposomes) if necessary. Such RNAs, vectors, and other necessary elements may be designed and produced according to anv conventional method.

Means for inhibiting the function of a translation product (protein) of a specific gene include, for example, compounds, antibodies (neutralizing antibodies), aptamers, or other inhibitors that inhibit the function by binding to the translation product, protein itself, or compounds, antibodies (neutralizing antibodies), aptamers, or other inhibitors that inhibit the function of the translation product, protein by binding to other proteins that interact with the translation product, protein (such as a receptor protein or a protein that, together with the translation product, forms a complex). Such inhibitors such as compounds, antibodies, or aptamers as described above can be administered to subject animal individuals by appropriate techniques (such as injection or immersion bath (addition into tanks)). The inhibitors to inhibit the function of a protein encoded by a specific gene, such as compounds, antibodies, or aptamers, may be known inhibitors (that have been observed to have inhibitory effect on the specific gene, etc. in different animals from the subject animal) or may be newly produced according to conventional methods of design, immunization, screening, and the like.

Means for enhancing the function of a specific gene itself include, for example, methods for overexpressing the specific gene by using an expression vector (such as a viral vector plasmid or an expression plasmid) into which the specific gene has been inserted or by inserting a high expression promoter into a promoter region for the specific gene in the genome or additionally integrating the specific gene into a particular region (target sequence) in the genome (e.g., by increasing the number of the specific gene in the genome from one to two or more) by genome editing as described above or other techniques. Suitable elements for these methods (such as RNA, proteins, mRNA, and expression vectors) can be designed and produced according to conventional methods and can be introduced into cells by administering the elements to individuals, embryos, eggs, or other cells of subject animals by using any suitable techniques (such as injection, electroporation, or addition into culture medium) in combination with a suitable means for delivering the elements into cells (such as liposomes) if necessary.

Means for enhancing the function of the transcription product (mRNA) of a specific gene include, for example, methods for administering the mRNA itself to individuals, embryos, eggs, and other cells of subject animals to allow translation of the mRNA in the cells by using any suitable techniques (such as injection, electroporation, or addition into culture medium), in combination with a suitable means for delivering the mRNA itself into cells (such as liposomes) if necessary. Such expression vectors, mRNA, and other necessary elements may be designed and produced according to anv conventional method.

Means for enhancing the function of a translation product (protein) encoded by a specific gene include, for example, methods for using compounds, antibodies (agonistic antibodies), aptamers, or other potentiators (agonists) that are responsible for the function equivalent to that of a translation product, protein by binding to other proteins that interact with the translation product, protein (such as a receptor protein or a protein that, together with the translation product, forms a complex). Such potentiators (agonists) such as compounds, antibodies, or aptamers as described above can be administered to subject animal individuals by appropriate methods (such as injection or immersion bath (addition into tanks)). The potentiators (agonists) including compounds, antibodies, and aptamers that are responsible for the function equivalent to that of a protein encoded by a specific gene may be known potentiators (agonists) (that have been observed to have enhancing effects on the specific gene, etc. in different animals from the subject animal) or may be newly produced according to conventional methods of design, immunization, screening, and the like.

How to perform the regulating (inhibiting and/or enhancing) step in subject animals is not particularly limited. The regulating step may be performed in an appropriate state (such as eggs, embryos, or individuals) or stage (such as molting stages of individuals) depending on the type of subject animals. When elements required for performing the regulating step are administered to subject animal individuals, the timing, number, interval, or site of administration may be appropriately determined in view of the effects of the present invention and the like. When the regulating is inhibition, examples of the elements include predetermined siRNAs or vectors to be used in RNA interference; elements to modify (such as delete) a specific gene in the genome by genome editing or other techniques, for example, predetermined RNAs, proteins, or vectors to be used in a CRISPR-Cas system; and inhibitors for a protein encoded by a specific gene or other proteins that interact therewith. When the regulating is enhancement, examples of the elements include expression vectors into which a specific gene has been inserted; elements to modify (such as insert) a specific gene, a promoter of the specific gene, or the like in the genome by genome editing or other techniques, for example, predetermined RNAs, proteins, or vectors to be used in a

CRISPR-Cas system; mRNA of a specific gene; and potentiators (agonists) for other proteins that interact with the specific gene. For example, such elements may be administered at any one time of before the first molting, before the second molting (between the first and second moltings), before the third molting (between the second and third moltings), or at other times in the life cycle of the subject animal or may be administered at multiple times, depending on a selected specific gene or the like. The elements may be administered in a single dose or in multiple doses at appropriate intervals per time, in view of the persistence and others of the effects of the present invention. The elements may be administered in a dosage (such as an amount of injection to the body or a concentration in a culture medium or a tank) that may be determined in view of the size and others of the individual so that a desired effect of regulating (promoting or suppressing) growth is achieved. The elements may be administered systemically or locally at any site of administration (via any route) depending on a site where a specific gene is expressed.

How much the function of a specific gene, etc. is regulated (inhibited and/or enhanced) is not particularly limited and may be controlled by using suitable means and conditions so that desired effects of the present invention are achieved. When the function of a specific gene, etc. is inhibited, for example, the specific gene may be knocked out, that is, the function of the specific gene, etc. may be completely inhibited (suppressed by 100%) by genome editing or other techniques or may be knocked down, that is, the function of the specific gene, etc. may be partially inhibited (suppressed by a percentage in the range of more than 0% to less than 100%) by genome editing, RNAi, antisense methods, or other techniques. For example, when an expression level of a transcription or translation product of a specific gene in a subject animal (treatment group) that has been subjected to the growth regulating method of the present invention is (1) statistically significantly decreased and/or (2) decreased to 1% or less, 5% or less, 10% or less, 20% or less, 30% or less, 40% or less, 50% or less, 60% or less, 70% or less, 80% or less, 90% or less, or 95% or less as compared with a subject animal (control group) that has not been subjected to the growth regulating (promoting or suppressing) method of the present invention, the function of the specific gene, etc. can be determined to be inhibited (i.e., to have achieved effects of the present invention in this embodiment). On the other hand, for enhancing the function of specific gene, etc., when an expression level of a transcription or translation product of a specific gene in a subject animal (treatment group) that has been subjected to the growth regulating method of the present invention is (1) statistically significantly increased and/or (2) increased to 110% or more, 120% or more, 140% or more, 150% or more, 160% or more, 180% or more, two-fold (200%) or more, 4-fold or more, 5-fold or more, 6-fold or more, 8-fold or more, 10-fold or more, or 100-fold or more as compared with a subject animal (control group) that has not been subjected to the growth regulating (promoting or suppressing) method of the present invention, the function of the specific gene, etc. can be determined to be enhanced (i.e., to have achieved effects of the present invention in this embodiment).

More directly, when the effect of a “growth promoting method of the present invention” or “growth suppressing method of the present invention” is achieved, it is only required that at least one of the following (1) or (2) is observed in at least any one of a period from administration to the initial subsequent (first) molting, a period between the first molting and the next (second) molting, a period between the second molting and the next (third) molting, or other periods. By way of example, when administration is made between the first and second molting in the life cycle of the subject animal, the “initial (first) molting subsequent to administration” refers to the second molting in the life cycle of the subject animal. As the following are observed at a later period after administration, the effect of the present invention can be evaluated to persist longer.

### <For Growth Promoting Methods>

(1) The number of days until molting (or between moltings) is statistically significantly decreased in the treatment group, as compared with the control group.
(2) The increasing rate of individual body weight and/or length (such as full length or carapace length) after molting (or between moltings) is statistically significantly elevated in the treatment group, as compared with the control group.

### <For Growth Suppressing Methods>

(1) The number of days until molting (or between moltings) is statistically significantly increased in the treatment group, as compared with the control group.
(2) The increasing rate of individual body weight and/or length (such as full length or carapace length) after molting (or between moltings) is statistically significantly decreased in the treatment group, as compared with the control group.

The growth regulating (promoting or suppressing) methods of the present invention can comprise a step except the regulating (inhibiting and/or enhancing) step as described above, if necessary. Such a step includes, for example, a step of culturing eggs or embryos of subject animals that have experienced the regulating step to confirm growth regulating effects to be obtained from the regulating step or to actually obtain subject animals that have been subjected to the growth regulating method and a step of rearing (including cultivating) subject animal individuals derived from such eggs or embryos or subjected to other regulating steps. Embodiments of such culturing, rearing (cultivating), or other steps are essentially similar to embodiments of such steps for common subject animals and may be performed by appropriately varying, for example, a dietary level and rearing density depending on regulated growth.

Subject Animals Subjected to Growth Regulating (Promoting or Suppressing) Methods

Animals belonging to the Decapoda according to the present invention (sometimes herein referred to as “method-experienced animals”) are subject animals that have been subjected to the growth regulating (promoting or suppressing) method of the present invention as described above (i.e., that have been obtained by subjecting the animals to the method) and have regulated (inhibited and/or enhanced) function of a specific gene, etc. (a specific gene or a transcription or translation product thereof).

The method-experienced animals of the present invention have characteristics that reflect the growth regulating (promoting or suppressing) method of the present invention to which the animals have been subjected. For example, when a means for inhibiting the function of a specific gene itself as described above (such as genome editing) is employed in the growth regulating method of the present invention, the method-experienced animals harbor a chromosome having a loss of function of the specific gene in the body. When a means for inhibiting the function of a transcription product (mRNA) of a specific gene (such as RNA interference) is employed in the growth regulating method of the present invention, the method-experienced animals harbor double-stranded RNA (siRNA) for suppressing the expression of the specific gene or other elements (such as a vector) for suppressing the expression of the specific gene, in the body. When a means for inhibiting the function of a translation product (protein) of a specific gene (such as RNA interference) is employed in the growth regulating method of the present invention, the method-experienced animals harbor an inhibitor, such as a compound, an antibody, or an aptamer, for inhibiting the function of the protein, in the body. On the other hand, when a means for enhancing the function of a specific gene itself as described above is employed in the growth regulating method of the present invention, the method-experienced animals harbor an expression vector for the specific gene, a chromosome into which a nucleotide sequence comprising the specific gene derived from the expression vector has been integrated, or a chromosome into which a high expression promoter for the specific gene has been inserted or the specific gene has been additionally integrated by genome editing or other techniques, in the body. When a means for enhancing the function of a transcription product (mRNA) of a specific gene is employed in the growth regulating method of the present invention, the method-experienced animals harbor (an overexpressed amount of) mRNA of the specific gene in the body. When a means for enhancing the function of a translation product (protein) of a specific gene is employed in the growth regulating method of the present invention, the method-experienced animals harbor a potentiator (agonist), such as a compound, an antibody, or an aptamer, that is responsible for the function equivalent to that of a protein encoded by the specific gene, in the body. It should be noted that the “in the body” may be “in cells” or “in the outside of cells” (for example, in the body fluid) of method-experienced animals depending on various embodiments as described above.

Such characteristic materials that are harbored by the method-experienced animals and have reflected the growth regulating (promoting or suppressing) method can be quantitatively or qualitatively detected according to any conventional methods. The method-experienced animals have inhibited or enhanced function of the specific gene, etc. As with the above description of the growth regulating methods of the present invention, how much the function of the specific gene, etc. is inhibited or enhanced is not particularly limited as long as any desired effects of the present invention can be achieved.

For example, when an individual (test individual) of animals belonging to the Decapoda has a statistically significantly decreased or increased expression level of a transcription or translation product of a specific gene as compared with a subject animal (control group or wild-type group) that is preferably in the same or similar lifecycle stage and molting cycle to the test individual and has not been subjected to the growth regulating (promoting or suppressing) method of the present invention, the test individual can be determined to be a method-experienced animal that has inhibited or enhanced function of the specific gene, etc., that is, in which the effects of the present invention have been achieved.

Particularly, the following (a) to (c) can be considered to be method-experienced animals of the present invention: (a) individuals, embryos, eggs, or the like having a chromosome that has not been reported to naturally (non-artificially) occur and which has a loss of function of a specific gene, or which integrates a specific gene, to a sufficient extent to cause a discernible effect of promoting or suppressing growth, (b) individuals having an artificially synthesized RNA (in some cases, including non-naturally occurring nucleic acids) such as an siRNA, a vector, or other nucleic acid molecules for inhibiting the function of mRNA of a specific gene or having mRNA for enhancing the function of mRNA of a specific gene, and (c) individuals having an inhibitor for inhibiting the function of a protein encoded by a specific gene, such as a compound, an antibody, or an aptamer or having a potentiator (agonist) for enhancing the function of a protein encoded by a specific gene, such as a compound, an antibody, or an aptamer.

### EXAMPLES

More specific embodiments of the growth regulating (promoting or suppressing) methods, method-experienced animals, and others of the present invention will be disclosed below in the Examples, but the technical scope of the present invention is not limited to the specific embodiments disclosed in the Examples. Those skilled in the art would understand that the embodiments disclosed in the Examples may be extended or modified into various other embodiments based on the description throughout the specification (including the drawings) and the technical idea of the present invention extracted therefrom or may be optionally further combined with technical features provided by the conventional technology (known inventions) or used in combination with the conventional technology (known inventions), in order to adapt the present invention to the intended use or effect.

### Example 1

In this Example, each gene of TSC2, PDK, Akt, FOXO, and p27 included in the specific genes (function-inhibiting group) and GFP gene (control group (GFP function-inhibiting group)) were knocked down by RNAi. The knockdown were performed using “dsTSC2”, “dsPDK”, “dsAkt”, “dsFOX0”, “dsp27”, and “dsGFP”, which are double strand (ds)RNAs targeting the following nucleotide sequences of TSC2, PDK, Akt, FOXO, p27, and GFP, respectively.

SEQ ID NO: 1: Target sequence for TSC2
5′-ATACAGCGAGCAATGCGAGTACTTGACCTTATGAGGCATCAAGAAA
CTCACAAAATAGGGGTATTGTATGTGGCTCAAAATCAAACTTCAGAACA
AGAAATTTTAAGGAATTCATGTGGTTCACTGCGTTACATGCATTTCCTT
CAGGGTTTAGGTACAGTCCTTGAGCTGAACTCTGTATCACAAGATGAAG
TATTCCTCGGTGGTCTTGACACCAAGGGTAACGATGGCAAGCTAG-3′
SEQ ID NO: 2: Target sequence for PDK
5′-AAACGATCGGACTTGGATTTTATCTTTGGCAAACTTATAGGAGAA
GGAAGTTTCTCAAGCGTTTACCTTGCAAAGGACATACACACAAATCAG
GAATATGCAGTTAAGGTTTGTGAAAAGCAGTTAATTATACGGGAGAAG
AAAGTGCAGCAAATAACCAGGGAAAGGGATGTAATGAACCTACTCAAC
AGCAACCAGAACCCTACGGCTCCGTTTTTCGTTAAACTTTCTTACGCC
TTCCAAGGAGA-3′
SEQ ID NO: 3: Target sequence for Akt
5′-GGTGGTCCAGGTGATGTAAGAGAGGTTCAGAGTCATCCCTTCTAT
GTAACAATCAACTGGAAACTTCTTGAAGAAAAAAAGTTAACTCCACCA
TTCAAGCCACAAGTAACCAGCGAGACTGACACCCGGTACTTCGATCGA
GAATTCACTGGAGAGTCTGTGCAGCTTACTCCACCTGATCAAGGGGAG
CACCTTAATGTTATTGATGAAGAATCAGAATACTTGACTTTCAACCAC
TTCTCTTATCAGGACATTTTATCAACTCTTGGCAGCTCACTAGCA-3′
SEQ ID NO: 4: Target sequence for FOXO
5′-CCCATGTCCCCTGGTATAGGTGGGTGGGGTGGCGAGTACTGGCCT
CACCATGCTCACCAACATCCACACCCGCACGACCGCTATGCCGACCAA
CTGGTAGACTCCATGGGGGAGGGACTCAAGCTAGGACCGGACTCTTGG
GGTGGCCCTGCTCGTCCGCCCAACCATCAGGACTGTATGAAACTATCC
CAGCTCTCCCC-3′
SEQ ID NO: 5: Target sequence for p27
5′-CAATGGCATGTTTGGATGATGAATACTCGTGGAGCCCGCCTTCAG
AAGCGGAACTCAAAGTTATTGAGGCCAGACGGGAACGTAACAACAAGA
TATCATCCATAATGGGACAATATCTTCTAAAGGGATACAAAATGTTGG
CTATAACATGCCCAGTTTGCGAGTGCATTTTGTTAGAGGATCGCATAC
AAAATAAATATTGCATCGGATGCAGTGAAGTTGATGCTGACACATGGA
AGGACAATCCAGCGGTTAGTGAAGAAGCAGCCAGAAGAGCAGTGGAAG
AAATTCA-3′
SEQ ID NO: 6: Target sequence for GFP (pAcGFP-N1)
5′-CACATGAAGCAGCACGACTTCTTCAAGAGCGCCATGCCTGAGGGC
TACATCCAGGAGCGCACCATCTTCTTCGAGGATGACGGCAACTACAAG
TCGCGCGCCGAGGTGAAGTTCGAGGGCGATACCCTGGTGAATCGCATC
GAGCTGACCGGCACCGATTTCAAGGAGGATGGCAACATCCTGGGCAAT
AAGATGGAGTACAACTACAACGCCCACAATG-3′

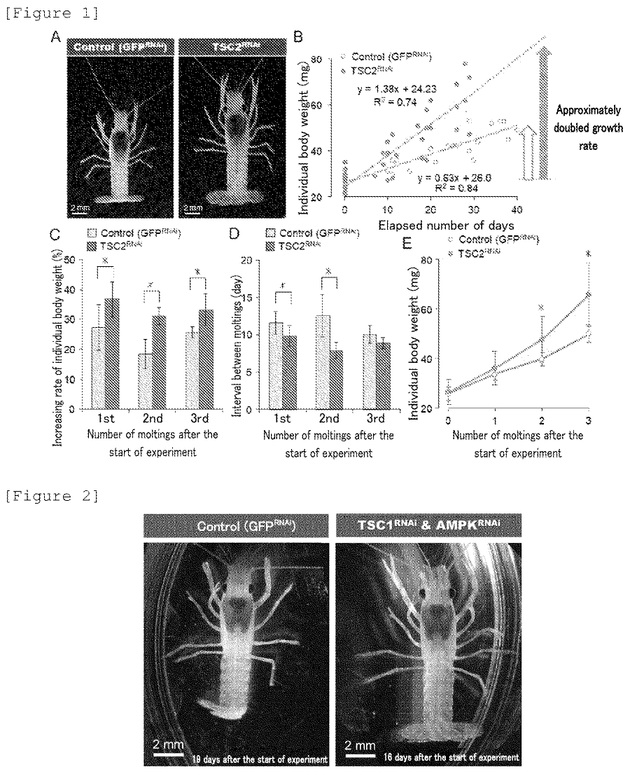

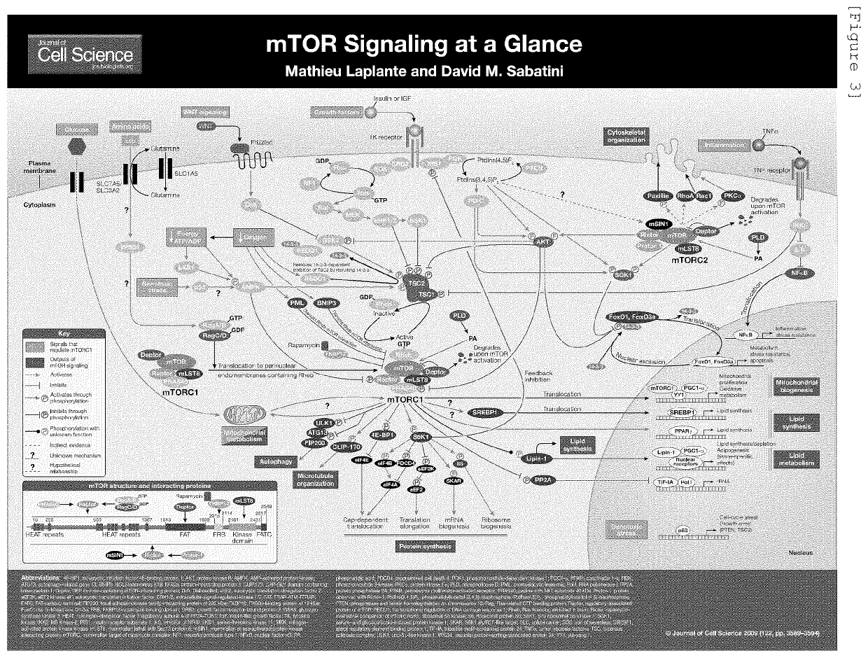

A predetermined number of marbled crayfish individuals in each group shown in Table 1 were injected with each of the dsRNAs described above and then observed for the progress of growth (the number of days until molting and increasing rate of individual body weight after molting) for about one month. The results are shown in Table 1 and FIG. 1. When the function of TSC2 was inhibited, the most stable growth-promoting effect was observed among the five genes in this Example (FIG. 1A). More specifically, inhibition of TSC2 function resulted in both promotion of molting and increase in the amount of growth per molting, and the resulting synergistic effect successfully increased the growth rate to about twice the normal rate (FIGS. 1B, C, and D). The results showed a remarkable increase in body size with a 32% increase in individual body weight relative to the normal rate after only three moltings (during a period of 5% or less of their lifetime in rearing) (FIG. 1E). Inhibition of TSC2 function is expected to ultimately result in an extremely high increase in body size in Decapoda crustaceans. Growth-promoting effects were also observed by inhibiting PDK, FOXO, and p27 functions. On the other hand, inhibition of Akt function resulted in increased number of days until molting and slower growth rates. This suggests that Akt may produce a growth-promoting effect on Decapoda crustaceans by conversely enhancing its function.

TABLE 1
Change of growth under conditions of knocking down specific
genes (TSC2, PDK, Akt, FOXO, and p27) by RNAi (n = 5-10)

| | | dsGFP | | | | | |
|---|---|---|---|---|---|---|---|
| | | (control) | dsTSC2 | dsPDK | dsAkt | dsFOXO | dsp27 |
| Amount of dsRNA | | 1 | 1 | 1 | 1 | 1 | 1 |
| injected (μg) | | | | | | | |
| 1st | Number of days | 11.6 ± 1.5 | 9.9 ± 1.4* | 10.4 ± 0.9 | 14.0 ± 1.7* | 8.7 ± 0.5* | 12.0 ± 0.8 |
| molting | until molting (day) | | | | | | |
| | Increasing rate of | 27.4 ± 7.8 | 36.8 ± 5.8* | 32.8 ± 6.2 | 22.6 ± 6.4 | 23.5 ± 9.9 | 14.1 ± 6.5* |
| | individual body weight | | | | | | |
| | after molting (%) | | | | | | |
| | Daily increasing rate of | 2.4 ± 0.9 | 3.6 ± 0.7* | 3.2 ± 0.8 | 1.7 ± 0.6 | 2.7 ± 1.2 | 1.2 ± 0.6* |
| | individual body weight | | | | | | |
| | (%/day) | | | | | | |
| | Ratio to control | 1.00 | 1.56 | 1.32 | 0.70 | 1.13 | 0.49 |
| 2nd | Number of days | 12.6 ± 2.9 | 7.9 ± 1.0* | 8.9 ± 0.9* | 12.3 ± 1.3 | 9.3 ± 2.2* | 9.7 ± 1.2* |
| molting | until molting (day) | | | | | | |
| | Increasing rate of | 18.5 ± 5.0 | 31.1 ± 2.7* | 34.5 ± 5.8* | 30.2 ± 4.7* | 26.5 ± 3.2* | 36.9 ± 2.7* |
| | individual body weight | | | | | | |
| | after molting (%) | | | | | | |
| | Daily increasing rate of | 1.5 ± 0.4 | 4.0 ± 0.7* | 3.9 ± 0.5* | 2.5 ± 0.6* | 3.0 ± 0.8* | 3.9 ± 0.6* |
| | individual body weight | | | | | | |
| | (%/day) | | | | | | |
| | Ratio to control | 1.00 | 2.68 | 2.60 | 1.69 | 2.02 | 2.60 |
| 3rd | Number of days | 10.0 ± 1.2 | 8.9 ± 0.8 | 9.8 ± 1.6 | 12.3 ± 1.2* | 8.8 ± 1.2 | 10.0 ± 0.8 |
| molting | until molting (day) | | | | | | |
| | Increasing rate of | 25.5 ± 2.0 | 33.2 ± 5.2* | 35.9 ± 5.9* | 26.2 ± 2.9 | 29.3 ± 4.8 | 28.7 ± 4.9 |
| | individual body weight | | | | | | |
| | after molting (%) | | | | | | |
| | Daily increasing rate of | 2.6 ± 0.4 | 3.7 ± 0.4* | 3.8 ± 1.0* | 2.1 ± 0.3 | 3.4 ± 0.9 | 2.9 ± 0.6 |
| | individual body weight | | | | | | |
| | (%/day) | | | | | | |
| | Ratio to control | 1.00 | 1.44 | 1.48 | 0.82 | 1.31 | 1.12 |

*P < 0.05 (Student’s t-test or Welch’s t-test)

Those skilled in the art would understand from the results of this Example that the effect of the present invention would be similarly achieved for TSC1 and TBC1D7, which form a complex with TSC2 to work. This is confirmed for TSC1 in the following Example 2.

### Example 2

dsRNAs were administered to marbled crayfish to knock down TSC1, AMPK, or both as in Example 1 except that the subject genes were changed to TSC1, AMPK, or both, and then the growth of the marbled crayfish was observed. The knockdown was performed using “dsTSC1” and “dsAMPK”, which are dsRNAs targeting the following nucleotide sequences of TSC1 and AMPK, respectively.

SEQ ID NO: 7: Target sequence for TSC1
5′-CCCTGAATCGACACCCTTTACTCACCAATAAAGATCGGAAGCCAG
TAANGTTGGCAGTCGCCGAACTGTTGCTGCATTGTGTAGCCTTAAAAC
TCAACACAAATGTAGGTAAGGACAACAGAGCCATCGTCTGCAGAGTTT
AGAGGCCAGTTCTAGGTCCTTTGTCACCTNTAAAAAGGAACAAACCCC
ATTCAGTTTTCCTGATCAGTGCCAAGACTTGTTTAATAGAGTGGAAGC
GATCTACCCTCCTCCAAAGTAAGTTGCAGC-3′
SEQ ID NO: 8: Target sequence for AMPK
5′-TCAAGATTCTCAACCGCAAAACTATCAAGAATTTGGATATGGTCA
GCAAGATAAAACGAGAAATAACAAATCTTAAATTGTTTCGTCATCCAC
ATATCATTAAACTGTACCAGGTGATCAGCACACCTACAGATATCTTTA
TGGTGATGGAATATGCTTCAGGAGGAGAGCTTTTTGACTATATTAAGA
AAAAAGGAAAGCTGAAGGAATCTGAAGCTCGCAGGTTCTT-3′

The results are shown in Table 2. When dsTSC1 and dsAMPK were used alone (1 μg) to knock down these genes, the growth-promoting effect was also observed. However, when both of these (0.5 μg each) were used together, the growth-promoting effect was observed at lower dosages. The TSC1/TSC2 complex is responsible for mediating the transmission of worsening environment, such as oxygen-deficient environment, downstream of the mTOR pathway, whereas AMPK is responsible for transmitting less energy available to cells downstream of the mTOR pathway. This suggests that inhibition of AMPK function produces the effect of giving the illusion that cells have available abundant energy, achieving growth promotion. It is also assumed that the double knockdown with TSC1 led to further growth promotion by giving the illusion that cells are in both the good environment and nutritional status. Those skilled in the art would understand from the results of this Example that knockdown of TSC2, instead of TSC1, together with AMPK may also result in a similar excellent growth-promoting effect.

TABLE 2
Change of growth under conditions of knocking down specific
genes (TSC1, AMPK, and both) by RNAi (n = 8-10)

| | | dsGFP | | | dsTSC1 |
|---|---|---|---|---|---|
| | | (control) | dsTSC1 | dsAMPK | & dsAMPK |
| Amount of dsRNA | | 1 | 1 | each10.5 | |
| injected (μg) | | | | | |
| 1st | Number of days | 10.6 ± 2.1 | 11.0 ± 0.9 | 10.4 ± 1.5 | 10.4 ± 1.7 |
| molting | until molting (day) | | | | |
| | Increasing rate of | 16.2 ± 4.7 | 23.9 ± 6.7 | 22.9 ± 5.5 | 28.5 ± 8.5* |
| | individual body weight | | | | |
| | after molting (%) | | | | |
| | Daily increasing rate of | 1.5 ± 0.2 | 2.2 ± 0.6* | 2.2 ± 0.5* | 2.8 ± 0.9* |
| | individual body weight | | | | |
| | (%/day) | | | | |
| | Ratio to | 1.00 | 1.45 | 1.46 | 1.86 |
| | control | | | | |
| 2nd | Number of days | 10.7 ± 0.9 | 7.7 ± 1.0* | 8.0 ± 0.6* | 7.6 ± 0.5* |
| molting | until molting (day) | | | | |
| | Increasing rate of | 19.9 ± 2.9 | 23.8 ± 7.5 | 19.7 ± 6.3 | 27.0 ± 9.3 |
| | individual body weight | | | | |
| | after molting (%) | | | | |
| | Daily increasing rate of | 1.9 ± 0.2 | 3.1 ± 1.1* | 2.2 ± 0.9 | 3.5 ± 1.3* |
| | individual body weight | | | | |
| | (%/day) | | | | |
| | Ratio to | 1.00 | 1.66 | 1.17 | 1.87 |
| | control | | | | |

*P < 0.05 (Student’s t-test or Welch’s t-test)

### Example 3

dsRNAs were administered to marbled crayfish to knock down PTEN and NIH as in Example 1 except that the subject genes were changed to PTEN and MIH, and then the growth of the marbled crayfish was observed. The knockdown was performed using “dsPTEN” and “dsMIH”, which are dsRNAs targeting the following nucleotide sequences of PTEN and MIH, respectively.

SEQ ID NO: 9: Target sequence for PTEN
5′-TGGCTACGACCTGGATCTCAGCTATATCACAGATCGTCTTATCGC
CATGGGCTTCCCTGCTCAGAAGTTGGAGGGTGTCTACAGAAACCATAT
TGATGACGTATGCCGCTTCCTAGAAGACAGACACAAGGACCATTATAG
AATATATAATTTGTGTTCTGAGAGAAATCGATCGTACGACGTAGCAAG
ATTCCATAACCGCGTTAGAACGTTCCCATTTGCTGACCACAATCCACC
TCCTCTGATTGATATCGAGCCACTATGCAAAGATATGGCAGATTGGCT
CAATGAAGATCAGAAAAATGTAGGCTGTTGTGCA-3′
SEQ ID NO: 10: Target sequence for MIH
5′-CCAGACCTGGAGAGGTTTCATACCTTAAGCTTGGTGCTGAGTTAC
CAGTAAGAGAAGAAGGTTCTACGAGTTGCTTGTGGAAGAGCACCAGAG
CGGGTGTCAGTAGTGCTTCAAGACATGGTTAACCAAGCTGCTCAATGC
TTCATTGTACGGAGAGTGTGGCTGGTGGTGGTGGTTGGGCTGCTGGTA
CACCAGACAGCGGCAAGGTATGTCTTCGAAGAATGTCCAGGAGTGATG
GGCAACCGAGCCGTCCACGGCAAGGTGACCCGGGTTTGTGAGGATTGC
TACAACGTCTTCAGGGACACTGAAGTCTTGGCTGGATGCAGGAAAGGC
TGCTTTTCTAGTGAGATGTTCAAGCTTTGCCTCTTGGCTATGGAGCGC
GTCGAGGAGTTTCCAGACTTCAAGAGATGGATTGGTATTCTTAACGCC
GGTC-3′

The results are shown in Table 3. The results of this Example showed that knockdown of only NIH led a trend toward shorter intervals between moltings and toward increased amounts of growth through molting, during the first molting. Knockdown of only PTEN significantly increased amounts of growth through molting during the first molting but significantly decreased amounts of growth through molting during the second molting. In contrast, knockdown of both PTEN and NIH resulted in no growth suppression during the second molting and provided a significant growth-promoting effect exceeding the effect obtained in knockdown of MIH or PTEN alone.

TABLE 3
Change of growth under conditions of knocking down specific
genes (PTEN, MIH, and both) by RNAi (n = 10-15)

| | | dsGFP | | | dsPTEN |
|---|---|---|---|---|---|
| | | (control) | dsPTEN | dsMIH | & dsMIH |
| Amount of dsRNA | | 1 | 1 | 1 | each 1 |
| injected (μg) | | | | | |
| 1st | Number of days | 11.5 ± 1.5 | 13.0 ± 3.5 | 10.1 ± 1.5* | 11.8 ± 3.2 |
| molting | until molting (day) | | | | |
| | Increasing rate of | 9.4 ± 5.6 | 16.7 ± 4.0* | 13.7 ± 6.3 | 17.2 ± 3.8* |
| | carapace length after | | | | |
| | molting (%) | | | | |
| | Daily increasing | 0.8 ± 0.5 | 1.4 ± 0.4* | 1.3 ± 0.6* | 1.6 ± 0.6* |
| | rate of carapace | | | | |
| | length (%/day) | | | | |
| | Ratio to | 1.00 | 1.62 | 1.52 | 1.91 |
| | control | | | | |
| 2nd | Number of days | 9.4 ± 1.2 | 10.0 ± 2.8 | 9.6 ± 1.2 | 9.3 ± 1.2 |
| molting | until molting (day) | | | | |
| | Increasing rate of | 11.8 ± 4.9 | 4.4 ± 4.1* | 12.2 ± 3.8 | 13.6 ± 5.9 |
| | carapace length after | | | | |
| | molting (%) | | | | |
| | Daily increasing | 1.2 ± 0.6 | 0.43 ± 0.4* | 1.3 ± 0.5 | 1.5 ± 0.6 |
| | rate of carapace | | | | |
| | length (%/day) | | | | |
| | Ratio to | 1.00 | 0.36 | 1.12 | 1.25 |
| | control | | | | |

### Example 4

Many quantitative characters, most notably growth, rarely involve only a single gene and often result from collective functions of multiple genes. For example, if a growth-promoting signal is increased, but another growth-suppressing signal surpasses the growth-promoting signal, the overall strength of the growth signal will be negative, probably leading to suppressed growth. Therefore, to identify genes involved in growth, it may be necessary to understand overall expression patterns of multiple genes and analyze relationship between the expression patterns and growth.

Following this idea, in Example 4, the present inventors first sampled juvenile crayfish littermates that were in the same molting cycle to obtain growth data and analyze the expression levels of candidate genes predicted to be associated with growth by real-time PCR. Exploiting the fact that the growth of crayfish is suppressed over time after the start of individual rearing, the present inventors sampled juvenile crayfish at multiple time points over time to obtain individuals with varying degrees of growth from genetically identical crayfish littermates. Focusing on mechanisms that integrate various cell growth and proliferation signals with environmental information at the cellular scale, candidate genes were selected mainly from upstream and downstream factors of the mTOR pathway and Akt pathway, including factors that have been demonstrated to be involved in growth regulation as shown in Examples 1 to 3.

Next, on the basis of the real-time PCR results, similar expression kinetics were summarized in the form of principal component scores by principal component analysis. The principal component scores are statistically estimated values that represent the overall expression patterns as described above. Finally, a model that can explain an association between the principal component scores and growth was established by means of a generalized linear model. Among the genes that had high principal component loadings for principal component scores correlated with growth, genes with direct or indirect transcriptional regulatory functions were presumed to be probably growth-related genes.

### [Rearing Experiment]

Juvenile marbled crayfish Procambarus virginalis from eggs spawned on the same day were used in the experiment. When molting was observed, individual body weights were measured the following day. Individuals having a body weight more than 20 mg were divided into two groups: one for observing the progress of growth and the other for analyzing gene expression. The former group was reared continuously, whereas eyestalks were collected from the latter group 3 days after molting and used as samples for RNA extraction.

### [RNA Extraction and Reverse Transcription]

Total RNA was extracted from the samples and reverse transcribed to synthesize cDNA. The synthesized cDNA was diluted 100-fold and used as a sample for subsequent real-time PCR analysis.

### [Real-Time PCR]

Real-time PCR was performed using commercially available real-time PCR reagents including SYBR Green and primers designed specifically for each gene. The primers used is listed in Table 4 below. For standard, all analytical samples were mixed together in equal volumes, and cDNA diluted 20- or 30-fold was further serially diluted 2-fold to be used. EF-1α was used as an internal control, and all measurements were quantified as relative values.

TABLE 4
List of primers used for real-time PCR in Example 4

| Gene name | Forward primer | SEQ ID No. | Reverse primer | SEQ ID No. |
|---|---|---|---|---|
| Akt | CCGTGACCTGCTTAAA [symbol] GTTTGTTA | 11 | TCTCTTACATCACCT [symbol] GACCACCTC | 12 |
| FOXO | TGAGTGAGAGTCTGGACCTGTACCC | 13 | GCTGAA [symbol] GCAGGGTG [symbol] ACTG | 14 |
| PDK | GCAAATAACCA [symbol] GGAAAG [symbol] GATGTA | 15 | AGTTTAACGAAAAACGGAGC [symbol] GTAG | 16 |
| P [symbol] 3K | ATT [symbol] CTACAAACACCGATCTGCCTA | 17 | CTCATCGTGTAATCTGTCCGTGATG | 18 |
| PTE [symbol] | AGCAAGATTCCATAACCG [symbol] GTTAGA | 19 | GATTGTG [symbol] TCAGCAAATGGGAAC | 20 |
| A [symbol] PK | ATTACCATCAACGAGAGCGACTTTG | 21 | GAGACTGACTGCTCAACGGCTTATC | 22 |
| mTO [symbol] | ACATTCACGCTTCAAGAGACCAAAG | 23 | GTCACCTTCTTGATTGTGGCTGAGT | 24 |
| P [symbol] AS40 | GGCATAGCAGAGGGACAGAAGTTTT | 25 | ATTTGGTGGCAGTTCAGACACTCTC | 26 |
| Raptor | AAGAATGGAGGCAGAAAGGCAGTAG | 27 | TCCACAGGCTCCAAGTTGAATACAT | 28 |
| R [symbol] eb | AGAAGCTGAAGGTTAGAGGGCAAGA | 29 | TCCAGCCGTGTCTACTAACTCCAAG | 30 |
| S [symbol] K1 | CGATAACGGTGATCCAATCAAGAAA | 31 | GGTTTAAATGGCGGTTCTAACTTGC | 32 |
| TSC1 | TGTGAGGACGTATGTGGCTCATTTT | 33 | ATTGGTGGACAAATTCAAAGGTTCC | 34 |
| TSC2 | GAACTATTCCTCGGTGGTCTTGACA | 35 | TACGTGGAAAACAACCTGCATAACC | 36 |
| 4 [symbol] BP | AGAACCTGCCAGTGATACCAGGAGT | 37 | CTCCTCTGAAATCGTTCCATTTTCC | 38 |
| FG [symbol] 1 | ACATCTACGCCATCCTGGAGGTTCG | 39 | AAGAGCCCAGCATCAACACCTCTGA | 40 |
| ILP | CACCTTGGCTGATATTCAAAGATGG | 41 | TTGCCAGAGATTGTTTGAGTTCTCC | 42 |
| [symbol] 7 [symbol] | CGGGAGGCACTTCATACATCTGTTA | 43 | AGTCAAACATCGAGTCCATCAGGAA | 44 |
| [symbol] cR | TCAAGAATGTCGCCTGAAGAAATGT | 45 | TTCTGTTCACGTTTTACCTGGCATT | 46 |
| Kr-h1 | TCGCCTCTTTCAAGTAATTCAGACG | 47 | ATATTGAGGCTGGTGAGGAGTTGGT | 48 |
| Met | TTGGCCACAGTCAGGTTGACTTATT | 49 | ATCGGGATGAATGACGTTGTAGATG | 50 |
| MI [symbol] | TTGCTACAACGTCTTCAGGGACA | 51 | CATAGCCAAGAGGCAAAGCTTGA | 52 |
| [symbol] F-1α | GCAGATTGTGCCGTGCTGAT | 53 | CTCGGGTCTGACCGTGCTT | 54 |

[symbol]

indicates data missing or illegible when filed

### [Analysis of Growth]

To examine gene expression at a given time and determine whether the gene expression contributes to growth, it is required to have been able to predict the most recent growth of an individual based on the state of the individual at that time. The state of individual can change in the rearing experiment, and thus an individual that has grown well will not always grow well after the next molting. In respect of growth, gene expression changes first, and then the change results in good or poor growth. Therefore, to screen for growth regulating genes, it is important to measure gene expression in individuals that are predicted with an accuracy above certain degree to exhibit good growth, rather than to measure gene expression in individuals that exhibit good growth after the gene expression have changed.

On the basis of this idea, in Example 4, a model was established to predict the growth through the next molting based on individual body weight on the day following the molting, the number of days from the start of individual rearing, and the number of moltings undergone, as described below. The growth of Decapoda crustaceans depends on the interval of moltings and the amount of growth per molting, and thus the present inventors first combined these into one variable as a principal component score by principal component analysis. Next, a model that predicts the principal component score based on the individual body weight on the day following the molting, the number of days from the start of individual rearing, and the number of moltings undergone at the time following the previous molting was established by means of a generalized linear model. The subsequent analysis was performed using R software version 4.0.2.

### [Analysis of Gene Expression Kinetics]

As noted above, it is generally believed that quantitative characters such as growth are rarely determined solely by the regulation of a single gene under naturally occurring conditions and are the collective results of the expression of many genes. On the basis of this idea, the behavior of genes with similar expression kinetics was converted into a principal component score by principal component analysis.

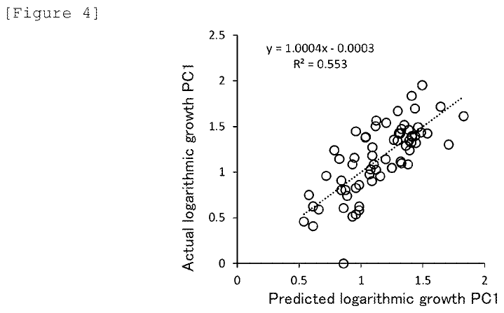

Prior to this analysis, putative ligand molecules that play a trigger role activating the Akt pathway and mTOR pathway via their receptors were searched as the upstream factors of these two pathways. A reciprocal BLAST search was performed to obtain genes homologous to wnt, EGF, FGF, and ILP as a candidate molecule from a gene catalog that had already been created, in reference to FIG. 4 and previous literatures with similar information. The results of quantification of expression from real-time PCR and principal component scores of growth principal component 1 were each approximated to a linear model by the least-squares method. Pearson product-moment correlation coefficients were calculated in these models and analyzed by t-test. The candidate molecules with significant correlation were considered to be promising ligand molecules that regulate the growth through the Akt pathway and mTOR pathway and were subjected to the principal component analysis described above.

### [Establishment of a Model Explaining the Relationship Between Growth and Gene Expression Kinetics]

Finally, a model explaining the principal component scores obtained from the analysis of growth by the principal component scores obtained from the analysis of gene expression was established by a generalized linear model using a Gaussian distribution with identity link function. Partial regression coefficients for each variable were analyzed by Wald test, and principal components with significant partial regression coefficients were considered to represent useful gene expression kinetics associated with growth regulation.

The correlation analysis performed prior to the principal component analysis in Example 4 showed that FGF1 and ILP had a significant positive correlation with the principal component score of growth principal component 1 (growth PC1), suggesting that FGFI and ILP are promising candidate molecules (FIG. 5A). Thus, the present inventors considered these two as upstream growth factors in the present invention and included them in subject factors analyzed in the principal component analysis.

For convenience in estimating and testing parameters, linearizing the distribution of data is an important process. A principal component score may take either a positive or negative value. For negative principal component scores, it is impossible to linearize a relationship that exhibits an exponential transition by logarithmic transformation. Therefore, in accordance with Zar (Data transformations. In: Biostatistical analysis. Prentice Hall, Englewood Cliffs, pp 236-243, 1984), a constant term was added, and then the sum was logarithmically transformed to perform parameter estimation in this study. The results are shown in Table 5.

TABLE 5
Parameters of the model that is estimated by a generalized
linear model and predicts a degree of post-molting
growth from a pre-molting state of an individual
as a principal component score of growth PC1

| | Partial regression | |
|---|---|---|
| | coefficient | |
| | (standard error) | |
| Intercept | 1.24 | (0.12)*** |
| Number of moltings M | −4.15 | (0.85)*** |
| Logarithmic individual body weight In W × number | 1.25 | (0.26)*** |
| of moltings M | | |
| Elapsed number of days D × number of moltings M | 0.18 | (0.03)*** |
| Logarithmic individual body weight In W × elapsed | −0.05 | (0.01)*** |

number of days D × number of moltings M

***P < 0.001 (Wald test)

The results can be used to estimate a post-molting logarithmic principal component score of growth PC1 and a post-molting principal component score of growth PC1 from a pre-molting state of an individual according to the equation:

ln (c−S_{PC1})=1.24−4.15 M+1.25 ln W·M+0.18 D·M−0.05 ln W·D·M

wherein S_{PC1} is the principal component score of growth PC1, M is the number of moltings after the start of individual rearing, W is the individual body weight, D is the number of days from the start of individual rearing, and c is a constant term that has its absolute value greater than the highest principal component score (in this case, the highest value +1). Thus, the principal component score S_{PC1} of growth PC1 is determined as follows:

S_{PC1}=−exp(1.24−4.15 M+1.25 ln W·M+0.18 D·M−0.05 ln W·D·M)+c

This model was judged to be the best based on the numerical values of AIC and coefficient of determination R² although several models were investigated, including with and without linearization by logarithmic transformation. The principal component scores S_{PC1} of growth PC1 obtained from the actual values are plotted against the S_{PC1} estimated from the rearing history using this model and were shown in FIG. 4. It is found that the quality of growth can be estimated with some accuracy based on the rearing history.

These results show that this model can be used to evaluate the extent of future growth in individuals sampled for gene expression analysis using principal component scores S_{PC1} of growth PC1 from the rearing history up to the sampling. The investigation of correlation between calculated principal component scores and the expression kinetics of each gene has identified genes that increase or decrease the expression in a growth-dependent manner.

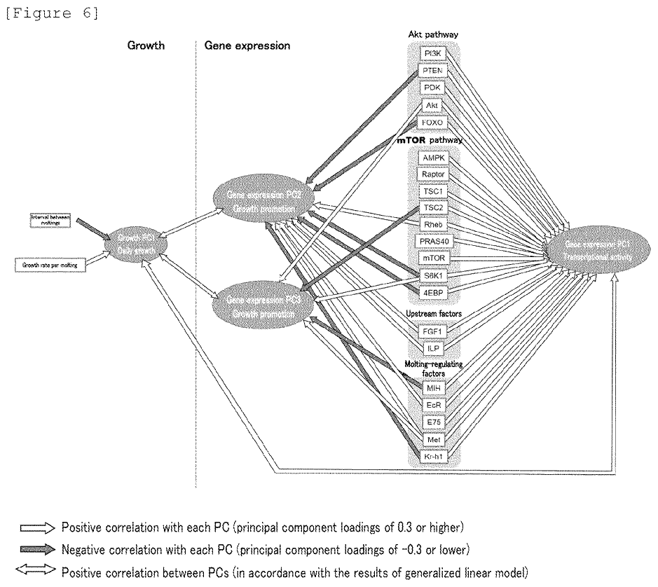

The results of the principal component analysis in Example 4 (principal component loadings and cumulative explanatory rate) are shown in Table 6, and the results of analyzing the principal component of gene expression correlated with growth PC1 are shown in Table 7. A path diagram model created on the basis of these results, which shows the relationship between the expression kinetics of each gene and growth, is shown in FIG. 6. The principal component 2 of gene expression (gene expression PC2) and gene expression PC3 having high standardized coefficients (Table 7) are shown to have a strong effect on growth. It has also been shown that the expression levels of various factors are positively (principal component loadings of 0.3 or higher) or negatively (principal component loadings of −0.3 or lower) correlated with gene expression PC2 and gene expression PC3. Factors that have expression levels shown to positively correlate with gene expression PC2 or PC3 have the effect of contributing growth promotion by enhancing the expression of the gene. Factors that have expression levels shown to negatively correlate have the effect of contributing growth promotion by inhibiting the expression of the gene. These factors can be understood as having the effect of the present invention of promoting growth alone or in combination with two or more of the factors. For example, the effect produced by using Akt (growth-suppressing effect by inhibiting its expression, that is, growth-promoting effect by enhancing its expression) and the effect produced by using FOXO and TSC2 (growth-promoting effect by inhibiting their expression) are as shown in Example 1. The effect produced by using MIH (growth-promoting effect by inhibiting its expression) is as shown in Example 3. The mTOR pathway includes molecules that are predicted to be rate-limiting factors highly correlated with growth. For example, Rheb highly correlates with growth and is expected to such a rate-limiting factor (FIG. 5B). In light of the fact that Rheb is suppressed by the TSC1/TSC2 complex, the results that growth is promoted by inhibiting TSC1, TSC2, or other factors by RNAi (Examples 1 and 2) are also consistent with the expectation that Rheb is a rate-limiting factor.

TABLE 6
Results of the principal component analysis of gene expression
kinetics under different growth conditions (principal component
loadings and cumulative explanatory rate)

| | | Principal component loading | | |
|---|---|---|---|---|
| | | Gene | Gene | Gene |
| Mechanism of | Gene | expression | expression | expression |
| target molecules | name | PC1 | PC2 | PC3 |
| Akt pathway | Akt | 0.755 | 0.056 | 0.530 |
| | FOXO | 0.598 | −0.344 | 0.146 |
| | PDK | 0.903 | −0.035 | −0.004 |
| | PI3K | 0.846 | 0.088 | 0.101 |
| | PTEN | 0.637 | −0.482 | 0.119 |
| mTOR pathway | AMPK | 0.880 | 0.041 | −0.168 |
| | mTOR | 0.840 | −0.292 | −0.240 |
| | PRAS40 | 0.918 | 0.154 | −0.078 |
| | Raptor | 0.847 | 0.093 | −0.262 |
| | Rheb | 0.632 | 0.490 | 0.203 |
| | S6K1 | 0.457 | −0.612 | 0.486 |
| | TSC1 | 0.927 | 0.022 | 0.141 |
| | TSC2 | 0.787 | 0.192 | −0.315 |
| | 4EBP | 0.646 | −0.604 | −0.204 |
| Growth factor | FGF1 | 0.552 | 0.443 | 0.042 |
| | ILP | 0.896 | 0.313 | 0.015 |
| Molting regulatory | E75 | 0.877 | −0.293 | −0.027 |
| factor | EcR | 0.768 | 0.445 | 0.097 |
| | Kr-h1 | 0.098 | −0.576 | 0.241 |
| | Met | 0.513 | 0.405 | 0.327 |
| | MIH | 0.750 | −0.208 | −0.510 |

| | | | |
|---|---|---|---|
| Cumulative contribution rate | 0.558 | 0.682 | 0.747 |

Principal component loadings having their absolute values of 0.3 or higher, which are generally considered to be a weak correlation, are indicated by boldface.

TABLE 7
Results of analyzing PC1 to 3 of gene expression
correlated with growth PC1 (standardized coefficients
estimated by a generalized linear model)

| | Standardized coefficients (standard error) | |
|---|---|---|
| Intercept | −0.673 | (0.129) *** |
| Gene expression PC1 | 0.315 | (0.131)* |
| Gene expression PC2 | 0.708 | (0.131)*** |
| Gene expression PC3 | 0.469 | (0.131)** |

*P < 0.05,
**P < 0.01,
***P < 0.001 (Wald test)

1-15. (canceled)

16. A method for regulating growth of an animal belonging to the Decapoda, comprising a step of regulating function of genes comprising:
at least one growth regulation-related gene selected from the group consisting of:
(i) a factor of mTOR pathway selected from the group consisting of AMPK, TBC1D, TSC1, TSC2 and Rheb,
(ii) a factor of Akt pathway,
(iii) an upstream factor of mTOR pathway and the Akt pathway, and
(iv) a downstream factor of mTOR pathway and Akt pathway; and
further optionally at least one molting-related gene selected from a molting-related factor, or function of a transcription or translation product of the genes.

17. The method according to claim 16, wherein the genes comprise at least one molting-related gene selected from the group consisting of EcR, Kr-hl, Met, and MIH.

18. The method according to claim 16, wherein the factor of Akt pathway is at least one selected from the group consisting of Akt, FOXO, PDK, and PTEN.

19. The method according to claim 16, wherein the downstream factor of mTOR pathway and Akt pathway is p27.

20. The method according to claim 16, wherein the upstream factor of mTOR pathway and Akt pathway is at least one selected from the group consisting of FGF1 and ILP.

21. The method according to claim 16, wherein the genes comprise, as the growth regulation-related gene, at least one selected from the group consisting of AMPK, TBC1D7, TSC1, TSC2, and PDK, and wherein the step comprises inhibiting the function of the growth regulation-related gene or a transcription or translation product thereof for regulation of the function, to promote the growth of the animal belonging to the Decapoda.

22. The method according to claim 21, wherein the growth regulation-related gene comprises at least AMPK and TSC1 and/or TSC2.

23. The method according to claim 16, wherein the genes comprise at least Akt as the growth regulation-related gene, and wherein the step comprises enhancing the function of the growth regulation-related gene or a transcription or translation product thereof for regulation of the function, to promote the growth of the animal belonging to the Decapoda.

24. The method according to claim 16, wherein the genes comprise at least Akt as the growth regulation-related gene, and wherein the step comprises inhibiting the function of the growth regulation-related gene or a transcription or translation product thereof for regulation of the function, to suppress the growth of the animal belonging to the Decapoda.

25. The method according to claim 16, wherein the genes comprise: at least PTEN as the growth regulation-related gene; and at least MIH as the molting-related gene, and wherein the step comprises inhibiting the function of the growth regulation-related gene and molting-related gene or a transcription or translation product thereof for regulation of the function, to promote the growth of the animals belonging to the Decapoda.

25. The method according to claim 16, wherein the regulating the function of the genes or the transcription or translation product thereof is to inhibit the function by suppressing the expression of the genes by RNA interference (RNAi), an antisense method, or genome editing.

26. An animal belonging to the Decapoda, having regulated function of genes comprising:
at least one growth regulation-related gene selected from the group consisting of:
(i) a factor of mTOR pathway selected from the group consisting of AMPK, TBC1D, TSC1, TSC2 and Rheb,
(ii) a factor of Akt pathway,
(iii) an upstream factor of mTOR pathway and the Akt pathway, and
(iv) a downstream factor of mTOR pathway and Akt pathway; and
further optionally at least one molting-related gene selected from a molting-related factor, or regulated function of a transcription or translation product thereof.

27. The animal according to claim 26, wherein the genes comprise at least one molting-related gene selected from the group consisting of EcR, Kr-hl, Met, and MIH.

28. The animal according to claim 26, wherein the factor of Akt pathway is at least one selected from the group consisting of Akt, FOXO, PDK, and PTEN.

29. The animal according to claim 26, wherein the downstream factor of mTOR pathway and Akt pathway is p27.

30. The animal according to claim 26, wherein the upstream factor of mTOR pathway and Akt pathway is at least one selected from the group consisting of FGF1 and ILP.

31. The animal according to claim 26, wherein the genes comprise, as the growth regulation-related gene, at least one selected from the group consisting of AMPK, TBC1D7, TSC1, TSC2, and PDK, and wherein the function of the growth regulation-related gene or a transcription or translation product thereof are inhibited for regulation of the function, to promote the growth of the animal belonging to the Decapoda.

32. The animal according to claim 31, wherein the growth regulation-related gene comprises at least AMPK and TSC1 and/or TSC2.

33. The animal according to claim 26, wherein the regulating the function of the genes or the transcription or translation product thereof is inhibition, and the animal harbors a double-stranded RNA or vector for suppressing the expression of the genes by RNA interference (RNAi), a chromosome having a loss of function of the genes, or an inhibitor for the translation product of the gene or a receptor thereof, in the body.
